(19) United States
(12) Patent Application Publication
(10) Pub. No.: US 2025/0290856 A1
(43) Pub. Date: Sep. 18, 2025

(54) DEVICE AND METHOD FOR HIGH-THROUGHPUT CHARACTERIZATION OF FLUIDS

(71) Applicants: Tao Li, Naperville, IL (US); Xingyl Lyu, DeKalb, IL (US); Kun Qian, Dongguan City (CN)

(72) Inventors: Tao Li, Naperville, IL (US); Xingyl Lyu, DeKalb, IL (US); Kun Qian, Dongguan City (CN)

(73) Assignee: Board of Trustees of Northern Illinois University, DeKalb, IL (US)

(21) Appl. No.: 19/081,343

(22) Filed: Mar. 17, 2025

Related U.S. Application Data
(60) Provisional application No. 63/566,373, filed on Mar. 17, 2024.

(51) Int. Cl. G01N 21/65; G01N 1/14; G01N 23/201; G01N 33/18

(52) U.S. Cl. CPC G01N 21/65; G01N 1/14; G01N 23/201; G01N 33/18

(57) ABSTRACT

A high-throughput system for spectroscopy of fluids includes (1) a first reservoir, (2) a fluid reservoir, (3) a first fluid connection, fluidly connecting the fluid reservoir and the first reservoir, (4) an examination chamber, (5) a second fluid connection, fluidly connecting the examination chamber and the first reservoir, (6) a third fluid connection, fluidly connecting the examination chamber and the first reservoir, and (7) at least one pump, configured to cause fluid in the first reservoir to flow into the examination chamber, and to cause fluid in the examination chamber to flow into the first reservoir.

### BACKGROUND

Aqueous electrolytes have been developed as an alternative to address the issues of the high flammability and volatility of organic electrolytes¹⁻³. However, the narrow electrochemical stability window limits the application of aqueous electrolytes in most electrochemical couples in lithium-ion batteries (LIBs)⁴⁻⁵. A new aqueous electrolyte called “water-in-salt” (WIS) containing 21 m (mol/kg) lithium bis(trifluoromethane sulfonyl) imide (TFSI), LiTFSI, has been proposed by Suo et al⁶. The large electrochemical stability window (˜3.0 V) of the WIS electrolyte is comparable to the organic electrolytes used in commercial LIBs. Salts consisting of TFSI anion show promise as aqueous electrolytes for LIBs, because of their relatively high chemical stability, low viscosity, and the ability to form stable solid-electrolyte interphase (SEI) to protect the electrolyte from a breakdown on the electrodes⁷⁻¹².

The solvation structure of LiTFSI aqueous electrolytes has been investigated with a variety of techniques¹³⁻²⁵. For example, Cho et al. found that bulk-like water molecules act as a medium for lithium-ion transport under 21 m LiTFSI aqueous electrolytes by combining femtosecond infrared spectroscopy (IR) and molecular dynamics (MD)¹⁷. With Fourier-transform infrared spectroscopy (FTIR) and MD simulation, Tokmakoff et al. revealed that at relatively high concentrations (larger than 3 m), Li cations contact with TFSI anions directly in the form of contact ion pairs, which have a significant population¹⁸. However, traditional techniques such as NMR¹⁶, Raman²⁶, and IR spectroscopy²⁷, are sensitive to the local molecular bonding and orientations and could not provide direct evidence of larger aggregates. Thus, we have used small-angle X-ray scattering (SAXS) to capture the whole structure of the forming clusters and aggregates²⁸. In our recent work, we found that there are two unique solvation structures in the LiTFSI aqueous solutions, TFSI-solvated structure and TFSI-network²⁹. For the WIS electrolyte containing 21 m LiTFSI, the TFSI-network is the only existing structure and is the key to the enlarged electrochemical stability window. Recently, Maginn's group has confirmed such structures with MD simulation and other scattering techniques^{30,35}. In addition, the two solvated structures were not only observed in LiTFSI, but also other TFSI salts with different cations. For example, NaTFSI aqueous solutions of different concentrations have structures similar to those of LiTFSI aqueous solutions²⁹. In addition to the monovalent lithium and sodium salt, we also found the TFSI-solvated structure and TFSI-network in the divalent magnesium salt magnesium bis(trifluoromethane sulfonyl) imide (Mg(TFSI)₂) solutions³¹.

Previous works mainly focus on the study of TFSI-based salts with the variation of cations. However, investigating the imide-based lithium salts aqueous solutions structures is insufficient³². We investigate the solvation behavior of four imide-based lithium salts, lithium bis(fluoro sulfonyl) imide (LiFSI), lithium bis(trifluoromethane sulfonyl)imide (LiTFSI), Lithium bis(pentafluoroethane sulfonyl) imide (LiBETI) and lithium bis (nonafluorobutane sulfonyl) imide (LiBNTI) aqueous solutions with different concentrations using SAXS experiments and MD simulations³³. We found that the anion solvated structures and anion networks also existed in these four imide-based lithium salts aqueous solutions. The anion solvated structure is the main structure at low concentrations, while the anion network dominates at the high concentration regime. From the SAXS experiments, the d spacing of anion solvated structure shows a linear relationship with the carbon numbers of the fluorocarbon chains and an exponential relationship with the concentration. From the MD simulation, we also found that the transition between these two characteristic structures is primarily controlled by the salt volume fraction (fₛₐₗₜ), which is defined as the effective volumetric ratio between the salt and water. This finding provides a quantitative estimation of the spatial occupation for both water and anions.

The techniques used to investigate the structure of these solvents, SAXS, as well as related techniques, such as WAXS, require the use of high intensity X-ray sources, for example the Advanced Photon Source (APS) 12-ID-B and 12-ID-C stations at Argonne National Laboratory, for which it can be challenging to schedule and obtain beam time. Even though the examination of any one sample is very fast, changing samples, and sample preparation, each the same except for solvent/solute concentrations, can be very time consuming. Furthermore, other spectroscopic techniques, such as Raman spectroscopy, can also be very time consuming to examine a large number of samples which otherwise only vary based on solvent/solute concentrations. Therefore, due to the laborious and time-consuming nature of conventionally configuring and characterizing solutions with gradient concentrations, achieving high data density has proven to be challenging.

### SUMMARY

In a first aspect, the invention is a high-throughput system for spectroscopy of fluids, comprising (1) a first reservoir, (2) a fluid reservoir, (3) a first fluid connection, fluidly connecting the fluid reservoir and the first reservoir, (4) an examination chamber, (5) a second fluid connection, fluidly connecting the examination chamber and the first reservoir, (6) a third fluid connection, fluidly connecting the examination chamber and the first reservoir, and (7) at least one pump, configured to cause fluid in the first reservoir to flow into the examination chamber, and to cause fluid in the examination chamber to flow into the first reservoir.

In a second aspect, the invention is a method for high-throughput spectroscopy using the high-throughput system, comprising simultaneously transferring the fluid from the fluid reservoir to the first reservoir; transferring the fluid from the first reservoir to the examination chamber; transferring the fluid from the examination chamber to the first reservoir, and examining the fluid in the examination chamber using a spectroscopic method. The method causes a concentration of the compound in the fluid within the examination chamber to increase or decrease.

### Definitions

Spectroscopy is the study of the absorption and emission of light and other radiation by matter.

### BRIEF DESCRIPTION OF THE DRAWINGS

FIG. 1 is a schematic of a high-throughput system for fluid characterization.

FIG. 2 is an illustration of a high-throughput system for fluid characterization.

FIG. 3 is photos of high-throughput systems for fluid characterization at different experimental sites: (a) a high-throughput system for SAXS at the 12-ID-B beamline of the Advanced Photon Source (APS) of Argonne National Laboratory (ANL), (b) a high-throughput system for SAXS at the 12-ID-C beamline of the APS of ANL, (c) a high-throughput system for WAXS at the Soft Matter Interface (SMI) beamline of the National Synchrotron Light Source II (NSLS-II) at Brookhaven National Laboratory, and (d) a high-throughput system for Raman spectroscopy at the Center for Nanoscale Materials (CNM) of ANL.

FIG. 4 is a graph of high-throughput SAXS data for the dilution process of a lithium bis (fluoro sulfonyl) imide (FSI) (LiFSI) aqueous solution (obtained at the 12-ID-C beamline).

FIG. 5 is a set of graphs comparing ex-situ(ES) and high-throughput (HT) SAXS data at various concentrations used to calibrate the high-throughput data for LiFSI aqueous solutions.

FIG. 6 is a 3-axis graph of the data from FIG. 4.

FIG. 7 is a graph of high-throughput SAXS data for the dilution process of a lithium bis(trifluoromethane sulfonyl) imide (TFSI) (LiTFSI) methanol solution (obtained at the 12-ID-C beamline).

FIG. 8 is a set of graphs comparing: ex-situ(ES) and high-throughput (HT) SAXS data at various concentrations used to calibrate the high-throughput data for LiTFSI methanol solutions.

FIG. 9 is a 3-axis graph of the data from FIG. 7.

FIG. 10 is a graph of high-throughput SAXS data for the dilution process of a lithium bis(pentafluoroethane sulfonyl) imide (BETI) (LiBETI) aqueous solution (obtained at the 12-ID-C beamline).

FIG. 11 is a set of graphs comparing: ex-situ(ES) and high-throughput (HT) SAXS data at various concentrations used to calibrate the high-throughput data for LiBETI aqueous solutions.

FIG. 12 is a 3-axis graph of the data from FIG. 10.

FIG. 13 is a graph of high-throughput WAXS data for the dilution process of a lithium bis (nonafluorobutane sulfonyl) imide (BNTI) (LiBNTI) aqueous solution (obtained at the SMI beamline).

FIG. 14 is a graph of high-throughput WAXS data for the dilution process of a lithium (pentafluoroethane sulfonyl) (trifluoromethane sulfonyl) imide (BETI/TFSI) (Li BETI/TFSI) (an asymmetric salt) aqueous solution (obtained at the SMI beamline).

FIG. 15 is a graph of high-throughput WAXS data for the dilution process of a lithium (nonafluorobutane sulfonyl) (trifluoromethane sulfonyl) imide (BNTI/TFSI) (LiBNTI/TFSI) (an asymmetric salt) aqueous solution (obtained at the SMI beamline SMI).

FIG. 16 is a graph of high-throughput Raman spectroscopy data for the dilution process of a LiTFSI aqueous solution.

FIG. 17 is a graph of high-throughput Raman spectroscopy data for the dilution process of a LiBETI aqueous solution.

FIG. 18 is a graph of high-throughput Raman spectroscopy data for the dilution process of a sodium bis (fluoro sulfonyl) imide (FSI) (NaFSI) aqueous solution.

FIG. 19 illustrates the structure of the anion of the asymmetric salt (pentafluoroethane sulfonyl) (trifluoromethane sulfonyl) imide (BETI/TFSI).

FIG. 20 illustrates the structure of the anion of the asymmetric salt (nonafluorobutane sulfonyl) (trifluoromethane sulfonyl) imide (BNTI/TFSI).

### DETAILED DESCRIPTION

In order to characterizing solutions with gradient concentrations, and achieving high data density quickly, a device and method has been developed. This innovative approach to in-situ characterization makes use of a single solution preparation, and through the implementation of dilution techniques, a significantly larger amount of data can be rapidly acquired compared to traditional non-in-situ characterization using spectroscopy. The method includes preparation of a high-concentration mixture placed in a sample reservoir, with circulation of the fluid through an examination chamber. Diluent is then introduced continuously into the sample reservoir, at a controlled rate to effect dilution. Simultaneously, a spectroscopic examination technique is applied to the fluid in the examination chamber, such as SAXS, WAXS, or Raman spectroscopy, at regular intervals to obtain data. This allows for the acquisition of large amounts of data, covering a large concentration range, to be acquired quickly.

Alternatively, a diluent may be placed in a reservoir, with circulation of the fluid through an examination chamber; and then a high-concentration mixture is introduced continuously into the reservoir, at a controlled rate to cause an increase in concentration. The diluent may be a liquid, and the mixture may be a solution of a solute in the liquid. Accordingly, in the description below, the sample reservoir and the fluid reservoir may be interchanged.

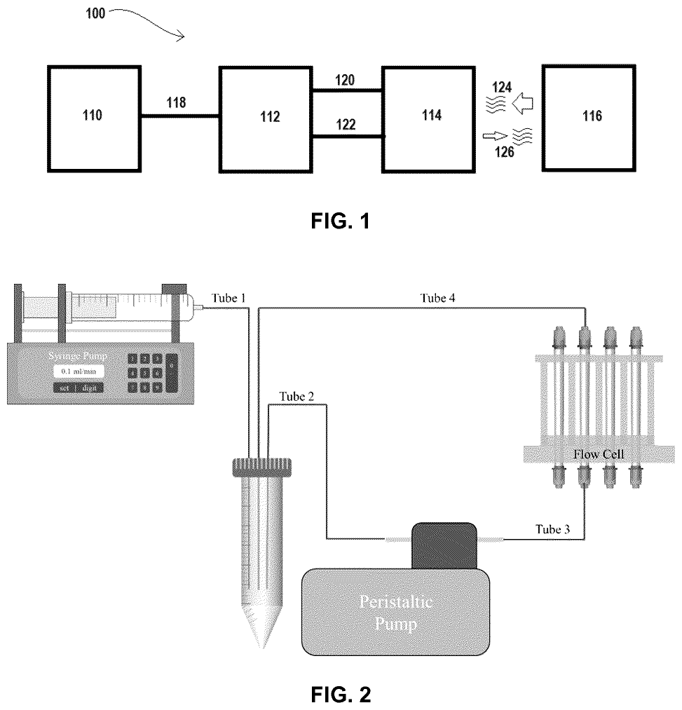

FIG. 1 is a schematic of a high-throughput system, 100, for fluid characterization. Shown in the figure are a fluid reservoir, 110, containing fluid for dilution of the sample, fluidly connected to a sample reservoir, 112, through fluid connection, 118, which will be continuously diluted by fluid from the fluid reservoir. Also shown is the examination chamber, 114, which is fluidly connected to the sample reservoir, and from which sample fluid flows to the examination chamber through a first fluid connection, 120, and then returned to the sample chamber from a second fluid connection, 122. Lastly, the schematic shows a spectroscopic device, 116, for spectroscopic examination of the fluid sample, using examination radiation, 124, and sample return radiation, 126, which comes from the sample. Although illustrated as the sample return radiation returning in the opposite direction as the examination radiation, in practice a variety of directions are possible depending on the type of spectroscopic technique used, and the location of the radiation source of the examination radiation and the detectors for receiving the sample return radiation. Not illustrated in the schematic is a pump for circulating the fluid between the sample reservoir and the examination chamber. Also not illustrated is an optional pump for transferring fluid from the fluid reservoir to the sample reservoir; such fluid transfer may also be carried out by gravitational flow if the fluid reservoir is placed at a height above the sample reservoir and the fluid is a liquid, rather than a gas.

FIG. 2 is an illustration of a high-throughput system for fluid characterization. Here, the fluid reservoir is a syringe containing solvent, such as water or methanol. The syringe is fluidly connected to the sample reservoir by Tube 1. A syringe pump is used to cause the solvent to flow from the syringe to the sample reservoir. In this system, the sample reservoir begins with a high concentration solution as the sample. A peristaltic pump is used to cause the sample solution from the reservoir to flow through Tube 2, through the peristaltic pump and into Tube 3, to the examination chamber which is a flow cell (two open-ended capillary tube) in this system, then return back to the sample chamber via Tube 4. In this illustration the examination chamber is a flow cell; as show there are several flow cells which allow for switching of Tubes 3 and 4 to different flow cells, thus avoiding the need to wash the flow cells between different measurements or series of measurements. This is particularly useful when expensive and/or limited beam time is available, to reduce the time needed for the series of high-throughput measurements.

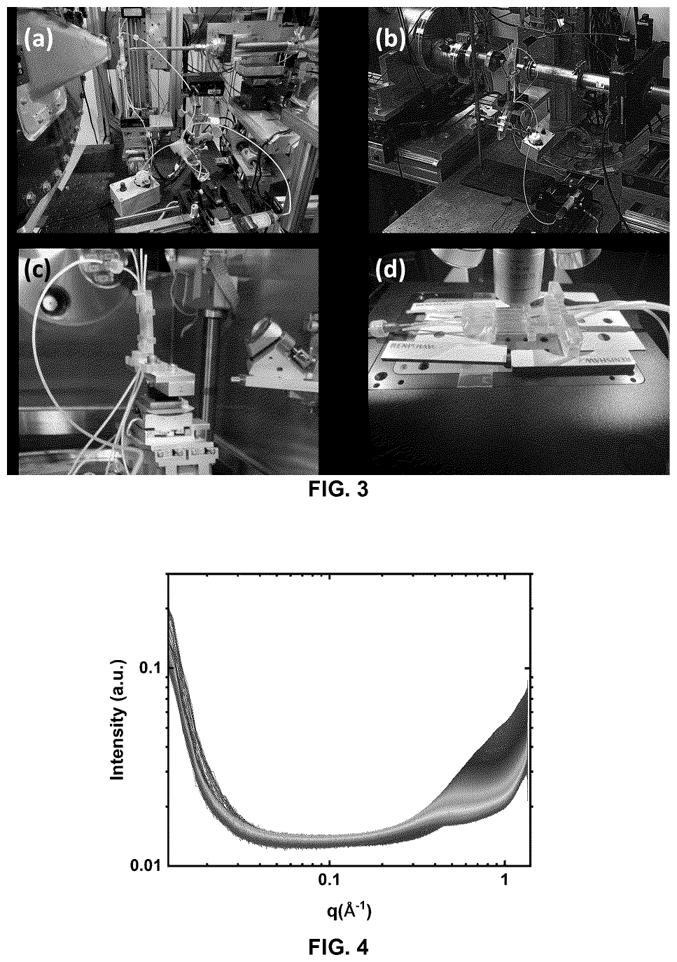

FIG. 3 is photos of high-throughput systems for fluid characterization used at different experimental sites. FIG. 3(a) is a high-throughput system for SAXS at the 12-ID-B beamline of the Advanced Photon Source (APS) of Argonne National Laboratory (ANL). FIG. 3(b) is a high-throughput system for SAXS at the 12-ID-C beamline of the APS of ANL. FIG. 3(c) is a high-throughput system for WAXS at the Soft Matter Interface (SMI) beamline of the National Synchrotron Light Source II (NSLS-II) at Brookhaven National Laboratory. FIG. 3(d) is a high-throughput system for Raman spectroscopy at the Center for Nanoscale Materials (CNM) of ANL.

Any type of spectroscopy may be used. In the examples, X-rays for SAXS and WAXS have been used, as well as high intensity monochromatic radiation for Raman spectroscopy, however any spectroscopy technique using any type of radiation could be used with the high-throughput system. In practice, the high-throughput system is most useful when scarce radiation sources, such as those available at US national labs, are used, to minimize the amount of time needed to obtain the desired data. Furthermore, although exemplified using liquids, gasses and supercritical fluids could also be used as the fluid.

### EXAMPLES

### Example 1: High-Throughput System for the Study of the Solvation Structure of Battery Electrolytes

A high-throughput system is illustrated in FIG. 2. There are three main modules in the entire set-up. The first one is the characterization module, a flow cell, which has a 3-D printed holder and 4 two-ends-open capillaries. The reason there are multiple capillaries inside is that, when changing the electrolyte, it can use other capillaries without the need for cleaning. This design helps save valuable beam time. The second main module is the dilution module, it contains a sample container, where the initial high-concentration electrolyte has been held and where the dilution process happens. A syringe loaded with the pure solvent was placed on a syringe pump which has remote control function. This function gives the experimental personnel the ability to start the pump and the measurement simultaneously from outside the hutch at the beamline. The third module is the circulation module, which contains a peristatic pump and several Teflon tubes. Tube 1 gets the solvent from the syringe, Tube 2 extracts the electrolyte from the sample container, Tube 3 pushes the electrolyte through the flow cell, and Tube 4 takes the electrolyte back to the sample container after being exposed to X-rays. In addition to the above function, this module also incorporates a mixing capability. The electrolyte in the Teflon tube, accelerated by the peristaltic pump, impacts the solutions in the sample container, facilitating the mixing process.

In this example, the following equipment or it equivalent was used: the syringe pump may be a NE-4000 Two Channel Programmable Syringe Pump from New Era. The speed used is 0.1 ml/minute, but this speed is adjustable. The syringe diameter was about 19.95 mm, with a 20 c.c. volume. The peristaltic pump may be an industrial peristaltic liquid pump with adjustable flowing dosing.

### Example 2: Study of LiFSI Aqueous Solution by SAXS using the High-Throughput System with the 12-ID-C Beamline

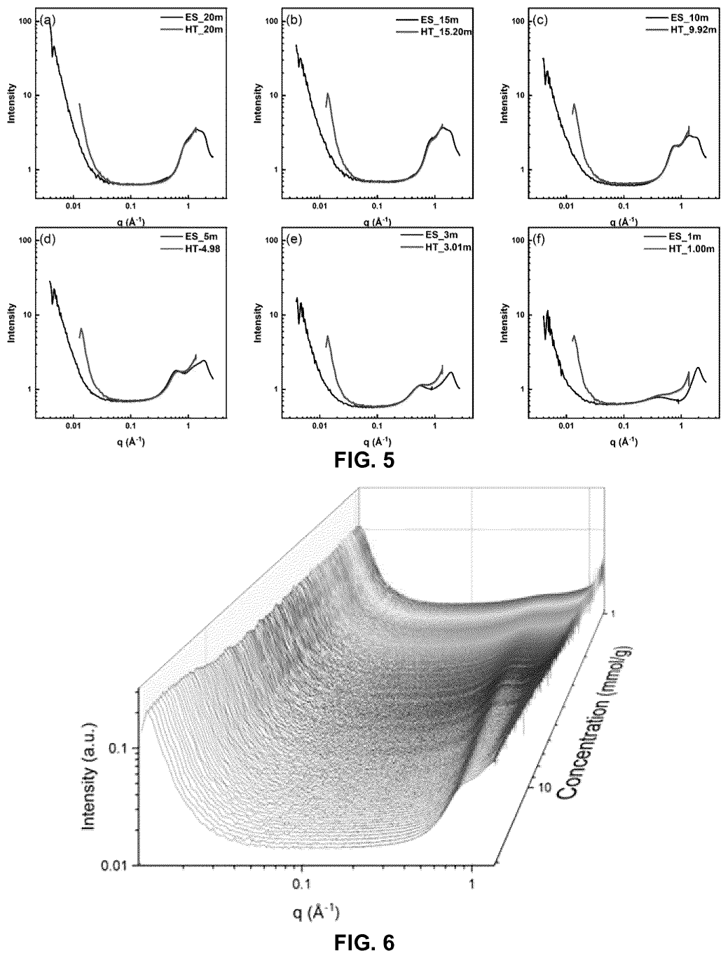

The high-throughput system shown in FIG. 3(b) was used to obtain SAXS data of an aqueous solution of LiFSI, over a concentration range of 20 mmol/g to about 1 mmol/g, at the 12-ID-C beamline. The data are shown in FIG. 4. 6 different ex situ samples were prepared of an aqueous solution of LiFSI, and SAXS data of each sample was obtain. The data from each of the ex situ samples was compared to the data obtain by the high-throughput system, and used to calibrate the concentration of the high-throughput samples, as shown in FIG. 5. After calibration, the high-throughput data was used to prepare the 3-axis graph shown in FIG. 6.

### Example 3: Study of LiTFSI Menthol Solution by SAXS using the High-Throughput System with the 12-ID-C Beamline

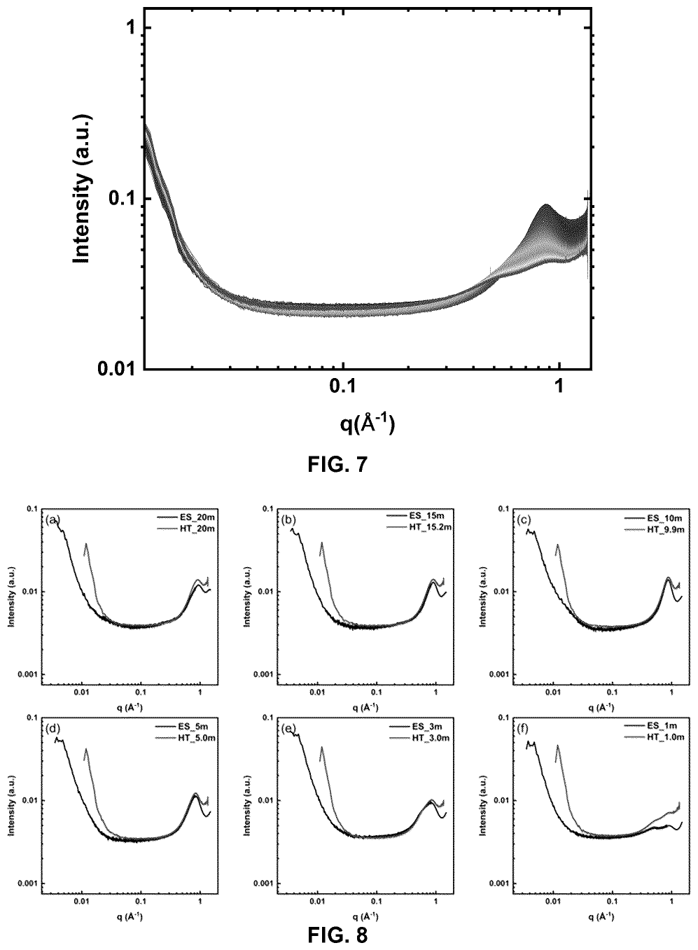

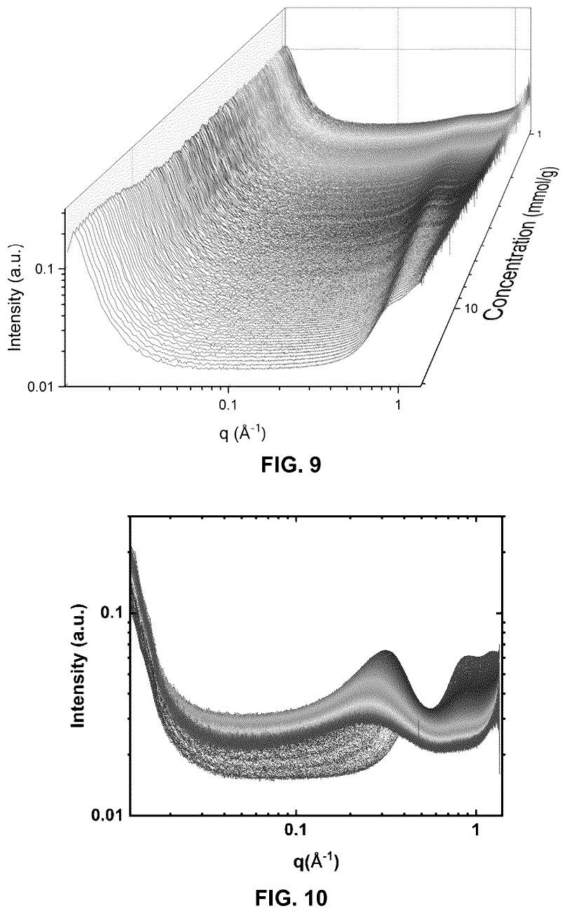

The high-throughput system shown in FIG. 3(b) was used to obtain SAXS data of a methanol solution of LiTFSI, over a concentration range of 20 mmol/g to about 1 mmol/g, at the 12-ID-C beamline. The data are shown in FIG. 7. 6 different ex situ samples were prepared of a methanol solution of LiTFSI, and SAXS data of each sample was obtain. The data from each of the ex situ samples was compared to the data obtain by the high-throughput system, and used to calibrate the concentration of the high-throughput samples, as shown in FIG. 8. After calibration, the high-throughput data was used to prepare the 3-axis graph shown in FIG. 9.

### Example 4: Study of LiBETI Aqueous Solution by SAXS using the High-Throughput System with the 12-ID-C Beamline

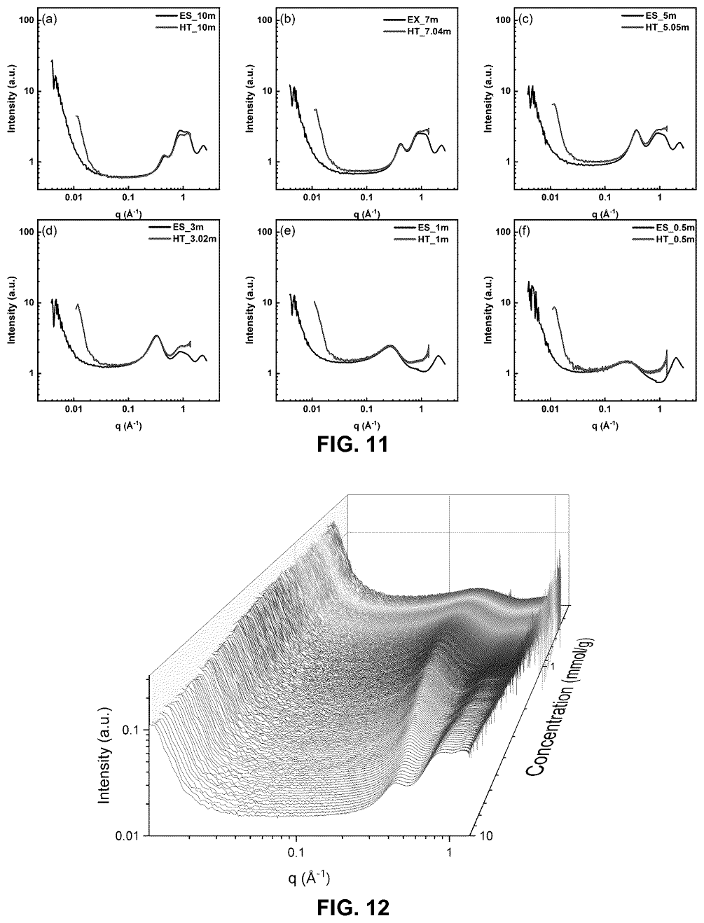

The high-throughput system shown in FIG. 3(b) was used to obtain SAXS data of an aqueous solution of LiBETI, over a concentration range of 10 mmol/g to about 0.5 mmol/g, at the 12-ID-C beamline. The data are shown in FIG. 10. 6 different ex situ samples were prepared of an aqueous solution of LiBETI, and SAXS data of each sample was obtain. The data from each of the ex situ samples was compared to the data obtain by the high-throughput system, and used to calibrate the concentration of the high-throughput samples, as shown in FIG. 11. After calibration, the high-throughput data was used to prepare the 3-axis graph shown in FIG. 12.

### Example 5: Study of LiBNTI Aqueous Solution by WAXS using the High-Throughput System with the SMI Beamline

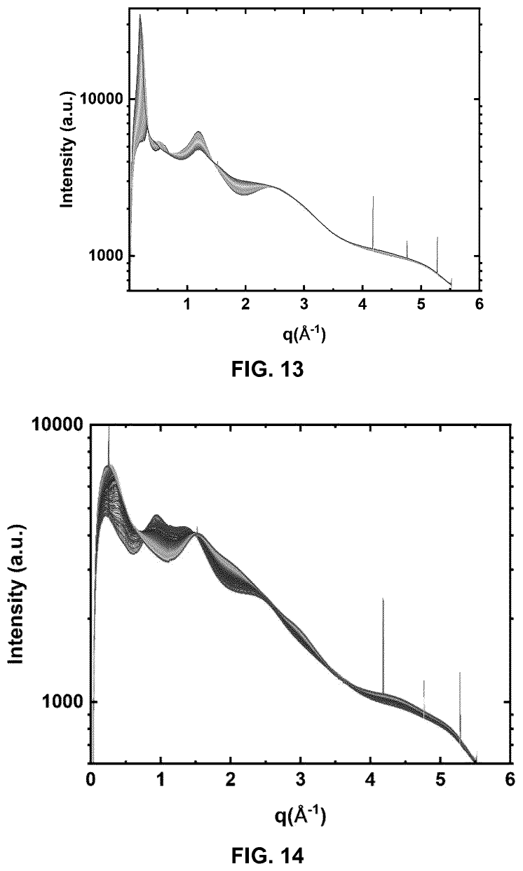

The high-throughput system shown in FIG. 3(c) was used to obtain WAXS data of an aqueous solution of LiBNTI, over a concentration range of 10 mmol/g to about 1 mmol/g, at the SMI beamline. The data are shown in FIG. 13.

### Example 6: Study of LiBETI/TFSI Aqueous Solution by WAXS using the High-Throughput System with the SMI Beamline

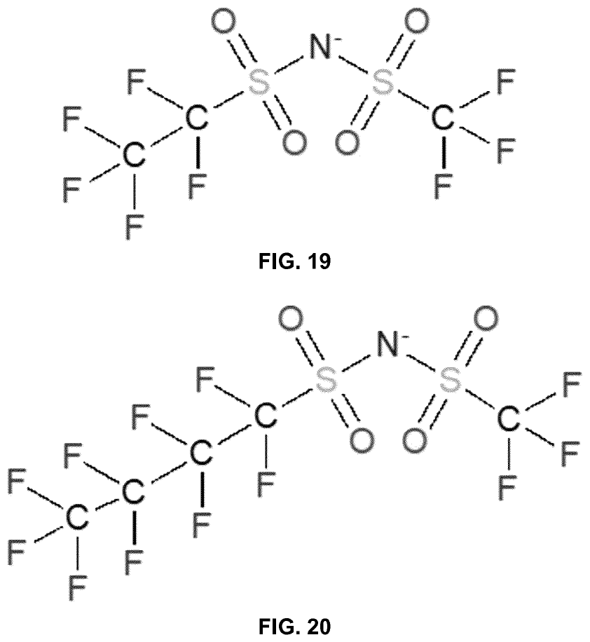

The high-throughput system shown in FIG. 3(c) was used to obtain WAXS data of an aqueous solution of LiBETI/TFSI, over a concentration range of 15 mmol/g to about 1 mmol/g, at the SMI beamline. The data are shown in FIG. 14. The structure of the anion of this asymmetric salt is shown in FIG. 19.

### Example 7: Study of LiBNTI/TFSI Aqueous Solution by WAXS using the High-Throughput System with the SMI Beamline

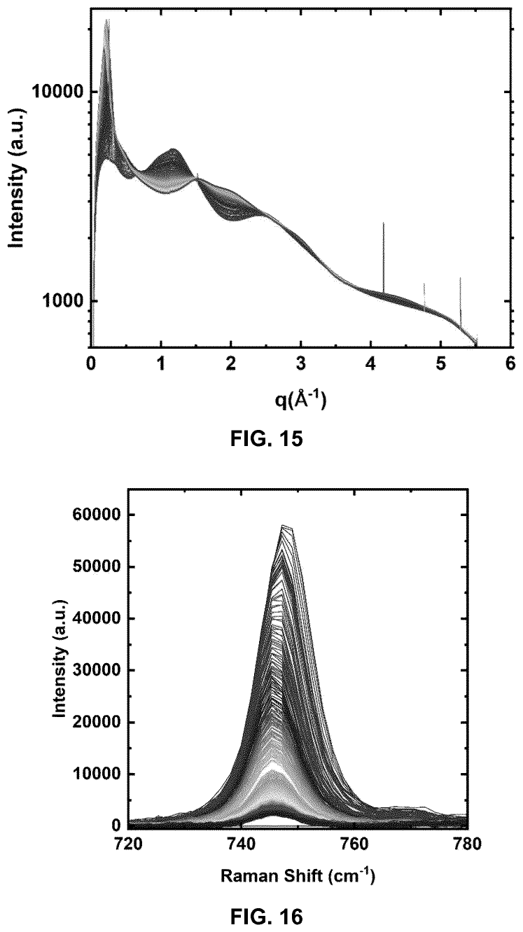

The high-throughput system shown in FIG. 3(c) was used to obtain WAXS data of an aqueous solution of LiBNTI/TFSI, over a concentration range of 15 mmol/g to about 1 mmol/g, at the SMI beamline. The data are shown in FIG. 15. The structure of the anion of this asymmetric salt in shown in FIG. 20.

### Example 8: Study of LiTFSI Aqueous Solution by Raman spectroscopy using the High-Throughput System with the CNM of ANL

The high-throughput system shown in FIG. 3(d) was used to obtain Raman spectroscopy data of an aqueous solution of LiTFSI, at the CNM of ANL. The data are shown in FIG. 16.

### Example 9: Study of LiBETI Aqueous Solution by Raman spectroscopy using the High-Throughput System with the CNM of ANL

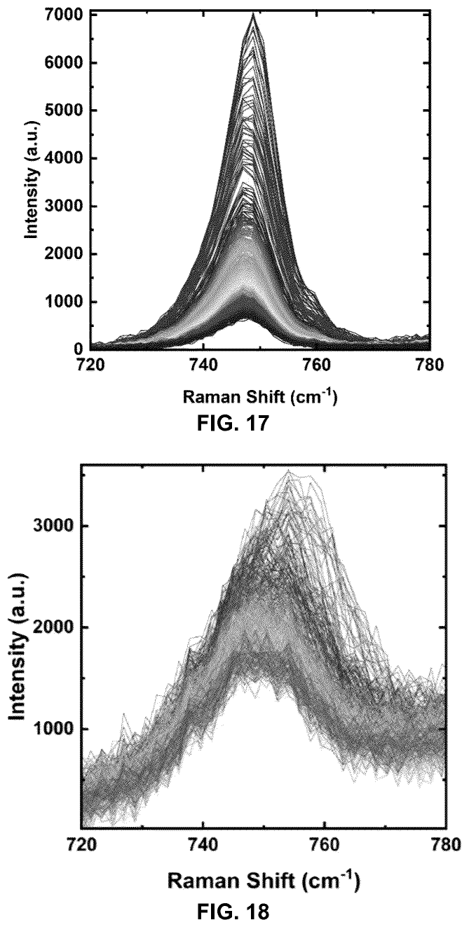

The high-throughput system shown in FIG. 3(d) was used to obtain Raman spectroscopy data of an aqueous solution of LiBETI, over a concentration range of 15 mmol/g to about 1 mmol/g, at the CNM of ANL. The data are shown in FIG. 17.

### Example 10: Study of NaFSI Aqueous Solution by Raman spectroscopy using the High-Throughput System with the CNM of ANL

The high-throughput system shown in FIG. 3(d) was used to obtain Raman spectroscopy data of an aqueous solution of NaFSI, at the CNM of ANL. The data are shown in FIG. 18.

### REFERENCES

1. Liu, Z.; Huang, Y.; Huang, Y.; Yang, Q.; Li, X.; Huang, Z.; Zhi, C., Voltage issue of aqueous rechargeable metal-ion batteries. Chem Soc Rev 2020, 49 (1), 180-232.
2. Alias, N.; Mohamad, A. A., Advances of aqueous rechargeable lithium-ion battery: A review. Journal of Power Sources 2015, 274, 237-251.
3. Armand, M.; Tarascon, J. M., Building better batteries. Nature 2008, 451 (7179), 652-7.
4. Wang, Y.; Yi, J.; Xia, Y., Recent Progress in Aqueous Lithium-Ion Batteries. Advanced Energy Materials 2012, 2 (7), 830-840.
5. Wessells, C.; Huggins, R. A.; Cui, Y., Recent results on aqueous electrolyte cells. Journal of Power Sources 2011, 196 (5), 2884-2888.
6. Suo, L.; Borodin, O.; Gao, T.; Olguin, M.; Ho, J.; Fan, X.; Luo, C.; Wang, C.; Xu, K., “Water-in-salt” electrolyte enables high-voltage aqueous lithium-ion chemistries. Science 2015, 350 (6263), 938-43.
7. Zhang, H.; Wang, D.; Shen, C., In-situ EC-AFM and ex-situ XPS characterization to investigate the mechanism of SEI formation in highly concentrated aqueous electrolyte for Li-ion batteries. Applied Surface Science 2020, 507.
8. Suo, L.; Oh, D.; Lin, Y.; Zhuo, Z.; Borodin, O.; Gao, T.; Wang, F.; Kushima, A.; Wang, Z.; Kim, H. C.; Qi, Y.; Yang, W.; Pan, F.; Li, J.; Xu, K.; Wang, C., How Solid-Electrolyte Interphase Forms in Aqueous Electrolytes. J Am Chem Soc 2017, 139 (51), 18670-18680.
9. Li, Z.; Jeanmairet, G.; Méndez-Morales, T.; Rotenberg, B.; Salanne, M., Capacitive Performance of Water-in-Salt Electrolytes in Supercapacitors: A Simulation Study. The Journal of Physical Chemistry C 2018, 122 (42), 23917-23924.
10. Wang, Z.; Li, X.; Chen, Y.; Pei, K.; Mai, Y.-W.; Zhang, S.; Li, J., Creep-Enabled 3D Solid-State Lithium-Metal Battery. Chem 2020, 6 (11), 2878-2892.
11. Li, X.; Chen, W.; Qian, Q.; Huang, H.; Chen, Y.; Wang, Z.; Chen, Q.; Yang, J.; Li, J.; Mai, Y. W., Electrospinning-Based Strategies for Battery Materials. Advanced Energy Materials 2020, 11 (2).
12. Chen, Y.; Wang, Z.; Li, X.; Yao, X.; Wang, C.; Li, Y.; Xue, W.; Yu, D.; Kim, S. Y.; Yang, F.; Kushima, A.; Zhang, G.; Huang, H.; Wu, N.; Mai, Y. W.; Goodenough, J. B.; Li, J., Li metal deposition and stripping in a solid-state battery via Coble creep. Nature 2020, 578 (7794), 251-255.
13. Yu, Z.; Curtiss, L. A.; Winans, R. E.; Zhang, Y.; Li, T.; Cheng, L., Asymmetric Composition of lonic Aggregates and the Origin of High Correlated Transference Number in Water-in-Salt Electrolytes. J Phys Chem Lett 2020, 11 (4), 1276-1281.
14. Yamada, Y.; Yamada, A., Review-Superconcentrated Electrolytes for Lithium Batteries. Journal of The Electrochemical Society 2015, 162 (14), A2406-A2423.
15. Vatamanu, J.; Borodin, O., Ramifications of Water-in-Salt Interfacial Structure at Charged Electrodes for Electrolyte Electrochemical Stability. J Phys Chem Lett 2017, 8 (18), 4362-4367.
16. Pauric, A. D.; Halalay, I. C.; Goward, G. R., Combined NMR and molecular dynamics modeling study of transport properties in sulfonamide based deep eutectic lithium electrolytes: LiTFSI based binary systems. Phys Chem Chem Phys 2016, 18 (9), 6657-67.
17. Lim, J.; Park, K.; Lee, H.; Kim, J.; Kwak, K.; Cho, M., Nanometric Water Channels in Water-in-Salt Lithium Ion Battery Electrolyte. J Am Chem Soc 2018, 140 (46), 15661-15667.
18. Lewis, N. H. C.; Zhang, Y.; Dereka, B.; Carino, E. V.; Maginn, E. J.; Tokmakoff, A., Signatures of lon Pairing and Aggregation in the Vibrational Spectroscopy of Super-Concentrated Aqueous Lithium Bistriflimide Solutions. The Journal of Physical Chemistry C 2020, 124 (6), 3470-3481.
19. Jeon, J.; Lee, H.; Choi, J.-H.; Cho, M., Modeling and Simulation of Concentrated Aqueous Solutions of LiTFSI for Battery Applications. The Journal of Physical Chemistry C 2020, 124 (22), 11790-11799.
20. Han, K. S.; Yu, Z.; Wang, H.; Redfern, P. C.; Ma, L.; Cheng, L.; Chen, Y.; Hu, J. Z.; Curtiss, L. A.; Xu, K.; Murugesan, V.; Mueller, K. T., Origin of Unusual Acidity and Li (+) Diffusivity in a Series of Water-in-Salt Electrolytes. J Phys Chem B 2020.
21. Galle Kankanamge, S. R.; Kuroda, D. G., Molecular Structure, Chemical Exchange, and Conductivity Mechanism of High Concentration LiTFSI Electrolytes. J Phys Chem B 2020, 124 (10), 1965-1977.
22. Borodin, O.; Suo, L.; Gobet, M.; Ren, X.; Wang, F.; Faraone, A.; Peng, J.; Olguin, M.; Schroeder, M.; Ding, M. S.; Gobrogge, E.; von Wald Cresce, A.; Munoz, S.; Dura, J. A.; Greenbaum, S.; Wang, C.; Xu, K., Liquid Structure with Nano-Heterogeneity Promotes Cationic Transport in Concentrated Electrolytes. ACS Nano 2017, 11 (10), 10462-10471.
23. Chandra Rath, P.; Wu, C.-J.; Patra, J.; Li, J.; Lee, T.-C.; Yeh, T.-J.; Chang, J. K., Hybrid electrolyte enables safe and practical 5 V LiNi0.5Mn1.5O4 batteries. Journal of Materials Chemistry A 2019, 7 (27), 16516-16525.
24. Rath, P. C.; Wang, Y.-W.; Patra, J.; Umesh, B.; Yeh, T.-J.; Okada, S.; Li, J.; Chang, J.-K., Composition manipulation of bis(fluorosulfonyl) imide-based ionic liquid electrolyte for high-voltage graphite//LiNi0.5Mn1.5O4 lithium-ion batteries. Chemical Engineering Journal 2021, 415.
25. Li, B.; Zhang, S.; Xia, F.; Huang, Y.; Ran, X.; Xia, Y.; Chen, Y.; Huang, W., Insights into the hole transport properties of LiTFSI-doped spiro-OMeTAD films through impedance spectroscopy. Journal of Applied Physics 2020, 128 (8).
26. Dou, Q.; Lei, S.; Wang, D.-W.; Zhang, Q.; Xiao, D.; Guo, H.; Wang, A.; Yang, H.; Li, Y.; Shi, S.; Yan, X., Safe and high-rate supercapacitors based on an “acetonitrile/water in salt” hybrid electrolyte. Energy & Environmental Science 2018, 11 (11), 3212-3219.
27. Seo, D. M.; Boyle, P. D.; Sommer, R. D.; Daubert, J. S.; Borodin, O.; Henderson, W. A., Solvate structures and spectroscopic characterization of LiTFSI electrolytes. J Phys Chem B 2014, 118 (47), 13601-8.
28. Qian, K.; Winans, R. E.; Li, T., Insights into the Nanostructure, Solvation, and Dynamics of Liquid Electrolytes through Small-Angle X-Ray Scattering. Advanced Energy Materials 2020, 11 (4), 2002821.
29. Liu, X.; Yu, Z.; Sarnello, E.; Qian, K.; Seifert, S.; Winans, R. E.; Cheng, L.; Li, T., Microscopic Understanding of the lonic Networks of “Water-in-Salt” Electrolytes. Energy Material Advances 2021, 2021, 7368420, 1-9.
30. Zhang, Y. and Maginn, E. J., Water-In-Salt LiTFSI Aqueous Electrolytes (2): Transport Properties and Li⁺ Dynamics Based on Molecular Dynamics Simulations. The Journal of Physical Chemistry B 2021 125 (48), 13246-13254.
31. Yu, Z.; Juran, T. R.; Liu, X.; Han, K. S.; Wang, H.; Mueller, K. T.; Ma, L.; Xu, K.; Li, T.; Curtiss, L. A.; Cheng, L., Solvation Structure and Dynamics of Mg (TFSI) 2 Aqueous Electrolyte. Energy & Environmental Materials 2021.
32. Mendez-Morales, T.; Li, Z.; Salanne, M., Computational Screening of the Physical Properties of Water-in-Salt Electrolytes **. Batteries & Supercaps 2020.
33. Liu X.; Lee, C.-L.; Seifer, S.; Winans, R. E.; Cheng, L.; Y Z; Li, T.; Insight into the nanostructure of “water in salt” solutions: A SAXS/WAXS study on imide-based lithium salts aqueous solutions. Energy Storage Materials 2022, 45, 696-703.
34. Wen, Z.; Fang, W.; Chen, L.; Guo, Z.; Zhang, N.; Liu, X.; Chen, G., Anticorrosive Copper Current Collector Passivated by Self-Assembled Porous Membrane for Highly Stable Lithium Metal Batteries. Advanced Functional Materials 2021, 31, 2104930.
35. Zhang, Y. et al., Water-in-Salt LiTFSI Aqueous Electrolytes (1): Liquid Structure from Combined Molecular Dynamics Simulation and Experimental Studies. The Journal of Physical Chemistry B 2021 125 (17), 4501-4513.
36. Xinyi Liu, Shao-Chun Lee, Soenke Seifer, Randall E. Winans, Lei Cheng, Y Z, Tao Li, Insight into the nanostructure of “water in salt” solutions: A SAXS/WAXS study on imide-based lithium salts aqueous solutions, Energy Storage Materials, Volume 45, 2022, 696-703.
37. Xinyi Liu, Shao-Chun Lee, Soenke Seifert, Lilin He, Changwoo Do, Randall E. Winans, Gihan Kwon, Y Z, and Tao Li, Revealing the Correlation between the Solvation Structures and the Transport Properties of Water-in-Salt Electrolytes, Chemistry of Materials 2023 35 (5), 2088-2094.
38. Hura, G., Menon, A., Hammel, M. et al. Robust, high-throughput solution structural analyses by small angle X-ray scattering (SAXS). Nat Methods 6, 606-612 (2009).
39. Kun Qian, Soenke Seifert, Randall E. Winans, and Tao Li, Understanding Solvation Behavior of the Saturated Electrolytes with Small/Wide-Angle X-ray Scattering and Raman Spectroscopy. Energy & Fuels 2021 35 (23), 19849-19855.

What is claimed is:

1. A high-throughput system for spectroscopy of fluids, comprising:
(1) a first reservoir,
(2) a fluid reservoir,
(3) a first fluid connection, fluidly connecting the fluid reservoir and the first reservoir,
(4) an examination chamber,
(5) a second fluid connection, fluidly connecting the examination chamber and the first reservoir,
(6) a third fluid connection, fluidly connecting the examination chamber and the first reservoir, and
(7) at least one pump, configured to cause fluid in the first reservoir to flow into the examination chamber, and to cause fluid in the examination chamber to flow into the first reservoir.

2. The high-throughput system of claim 1, wherein:
the first reservoir contains a compound in a fluid, and
The fluid reservoir contains the fluid without the compound.

3. The high-throughput system of claim 1, further comprising a spectrometer configured to examine fluid in the examination chamber.

4. The high-throughput system of claim 3, wherein the spectrometer comprises a Raman spectrometer, a beamline for producing high intensity X-rays, or both.

5. The high-throughput system of claim 1, wherein the examination chamber comprises a two-ends-open capillary tube.

6. The high-throughput system of claim 1, wherein:
the first fluid connection comprises a tube,
the second fluid connection comprises a tube, and
the third fluid connection comprises a tube.

7. The high-throughput system of claim 1, further comprising a fluid reservoir pump, configured to cause fluid in fluid reservoir to flow into the first reservoir.

8. The high-throughput system of claim 1, wherein:
the fluid reservoir comprises a syringe,
the first fluid connection comprises a tube,
the examination chamber comprises a two-ends-open capillary tube,
the second fluid connection comprises a tube,
the third fluid connection comprises a tube,
the at least one pump comprises a peristaltic pump, and
the system further comprising a syringe pump configured to cause the fluid in the
fluid reservoir to flow into the first reservoir.

9. The high-throughput system of claim 3, wherein:
the fluid reservoir comprises a syringe,
the first fluid connection comprises a tube,
the examination chamber comprises a two-ends-open capillary tube,
the second fluid connection comprises a tube,
the third fluid connection comprises a tube,
the at least one pump comprises a peristaltic pump, and
the spectrometer comprises a Raman spectrometer, a beamline for producing high intensity X-rays, or both.

10. The high-throughput system of claim 2, wherein the fluid is a liquid.

11. The high-throughput system of claim 2, wherein the compound is a solute dissolved in the fluid.

12. The high-throughput system of claim 10, wherein the compound is a solute dissolved in the liquid.

13. The high-throughput system of claim 12, wherein the liquid comprises water and/or an alcohol, and the solute is a salt.

14. The high-throughput system of claim 13, wherein the salt is a salt comprising at least one member selected from the group consisting of lithium, sodium, magnesium, and calcium.

15. A method for high-throughput spectroscopy using the high-throughput system of claim 2, comprising simultaneously:
transferring the fluid from the fluid reservoir to the first reservoir,
transferring the fluid from the first reservoir to the examination chamber,
transferring the fluid from the examination chamber to the first reservoir, and
examining the fluid in the examination chamber using a spectroscopic method,
wherein the method causes a concentration of the compound in the fluid within the examination chamber to increase or decrease.

16. The method of claim 15, wherein the spectroscopic method is SAXS, WAXS or Raman spectroscopy.

17. The method of claim 16, wherein the spectroscopic method is SAXS or WAXS, and the method is carried out using a beamline of high intensity X-rays.

18. The method of claim 15, wherein the fluid is a liquid, and the compound is a solute dissolved in the liquid.

19. The method of claim 18, wherein the liquid comprises water and/or an alcohol, and the solute is a salt.

20. The method of claim 19, wherein the salt is a salt comprising at least one member selected from the group consisting of lithium, sodium, magnesium, and calcium.
